(19) United States
(12) Patent Application Publication
(10) Pub. No.: US 2024/0122881 A1
(43) Pub. Date: Apr. 18, 2024

(54) COMBINATION FOR USE IN TREATING CANCERS

(71) Applicant: SpringWorks Therapeutics, Inc., Stamford, CT (US)

(72) Inventor: Todd SHEARER, Stamford, CT (US)

(21) Appl. No.: 18/487,237

(22) Filed: Oct. 16, 2023

Related U.S. Application Data
(60) Provisional application No. 63/379,617, filed on Oct. 14, 2022.

(51) Int. Cl. A61K 31/165; A61K 31/4375

(52) U.S. Cl. CPC A61K 31/165; A61K 31/4375

(57) ABSTRACT

The present disclosure relates to methods of treating cancers comprising co-administration of mirdametinib (Compound A) or a pharmaceutically acceptable salt thereof, and 1-((1S,1aS,6bS)-5-((7-oxo-5,6,7,8-tetrahydro-1,8-naphthyridin-4-yl)oxy)-1a,6b-dihydro-1H-cyclopropa[b]benzofuran-1-yl)-3-(2,4,5-trifluorophenyl) urea (Compound B) or a pharmaceutically acceptable salt thereof, to a patient in need thereof.

### CROSS-REFERENCE TO RELATED APPLICATIONS

The present application claims priority to U.S. Provisional Application No. 63/379,617 filed Oct. 14, 2022, which is hereby incorporated by reference in its entirety.

### FIELD OF THE INVENTION

The present disclosure relates to methods of treating cancers comprising co-administration of Compound A or a pharmaceutically acceptable salt thereof, and Compound B or a pharmaceutically acceptable salt thereof, to a patient in need thereof. Also disclosed are pharmaceutical compositions of Compound A or a pharmaceutically acceptable salt thereof, and Compound B or a pharmaceutically acceptable salt thereof, for use in such cancer treatment. The disclosure further pertains to use of combinations of Compound A or a pharmaceutically acceptable salt thereof, and Compound B or a pharmaceutically acceptable salt thereof, for the manufacture of medicaments for cancer treatment.

### BACKGROUND

Certain cancers have mutations in the mitrogen-activated protein kinase (MAPK) signaling pathway leading to hyper-activation and proliferation of the mutated cells. Although FDA has approved kinase inhibitors that target certain mutations, relapses do occur. Therefore, there is a continued need for regimens to treat cancers.

### BRIEF SUMMARY OF THE INVENTION

Methods of treating a patient having a solid tumor comprising co-administering to the patient a therapeutically effective amount of Compound A, or a pharmaceutically acceptable salt thereof, and a therapeutically effective amount of Compound B, or a pharmaceutically acceptable salt thereof are disclosed herein. In some aspects, the therapeutically effective amount of Compound A is about 1 mg to about 10 mg per day. In some aspects, the therapeutically effective amount of Compound B, or a pharmaceutically acceptable salt thereof, is about 2 mg to about 50 mg per day. In some aspects, the therapeutically effective amount of Compound A is a total amount of up to 10 mg per day and the therapeutically effective amount of Compound B, or a pharmaceutically acceptable salt thereof, is about 5 mg to about 40 mg per day. In some aspects, the therapeutically effective amount of Compound A is administered in two equal doses.

In certain aspects, Compound B is a pharmaceutically acceptable salt form. In some aspects, the pharmaceutically acceptable salt form is hydroxypropyl methyl cellulose acetate succinate.

In other aspects, Compound A, or pharmaceutically salt form, and Compound B, or a pharmaceutically acceptable salt thereof, are administered in the same or different dosage forms.

In yet other aspects, Compound A is administered in free base form.

In some aspects, Compound B, or a pharmaceutically acceptable salt thereof, is administered once per day.

In other aspects, Compound A, or a pharmaceutically acceptable salt thereof, is administered before, concomitantly, or subsequently to the administering of Compound B, or a pharmaceutically acceptable salt thereof.

In certain aspects, Compound A, or a pharmaceutically acceptable salt thereof, and Compound B, or a pharmaceutically acceptable salt thereof, are administered orally.

In still yet other aspects, the solid tumor is selected from the group consisting of malignant peripheral nerve sheath tumor, biliary tract cancer, breast cancer, cholangiocarcinoma, urothelial cancer, uterine neoplasm, gastric cancer, sarcoma, bladder cancer, head and neck cancer, endometrial cancer, esophageal cancer, adenoid cystic carcinoma, gallbladder cancer, colorectal cancer, thyroid cancer, hepatocellular cancer, prostate cancer, oral cancer, cervical cancer, pancreatic carcinoma, ovarian cancer, melanoma, lung cancer, and serous carcinoma to the peritoneum. In some aspects, the lung cancer is selected from the group consisting of lung adenocarcinoma, squamous non-small cell lung cancer, non-squamous non-small cell lung cancer, and small cell lung cancer. In certain aspects, the patient has non-small cell lung cancer. In some aspects, the patient has non-small cell lung cancer (NSCLC) that is NSCLC with BRAF Class II/III/fusion mutations or KRAS mutant NSCLC.

In certain aspects, the patient has melanoma. In some aspects, the patient has melanoma that is NRAS mutant cutaneous melanoma.

In some aspects, the patient has endometrial cancer. In other aspects, the patient has ovarian cancer. In yet other aspects, the patient has low grade serous ovarian cancer.

In some aspects, the patient has a confirmed mutation in one or more of KRAS, NRAS, HRAS, BRAF, NF1, MEK1, and MEK2.

In certain aspects Compound A, or a pharmaceutically acceptable salt thereof, and Compound B, or a pharmaceutically acceptable salt thereof, are administered on a 28-day dosing cycle comprising administering Compound A, or a pharmaceutically acceptable salt thereof, twice a day and Compound B, or a pharmaceutically acceptable salt thereof, will be administered once per day. In some aspects, the 28-day dosing cycle is repeated up to a total of 24 consecutive 28-day dosing cycles.

In some aspects, Compound A, or pharmaceutically acceptable salt thereof, and/or Compound B, or pharmaceutically acceptable salt thereof, are administered in tablet and/or capsule form.

### Definitions

To facilitate understanding of the disclosure set forth herein, a number of terms are defined below.

Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs.

In this specification and the appended claims, the singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “Compound A” refers to the single enantiomer N-[(2R)-2,3-dihydroxypropoxy]-3,4-difluoro-2-(2-fluoro-4-iodoanilino)benzamide (mirdametinib).

The term “Compound B” refers to the compound having the following structure:

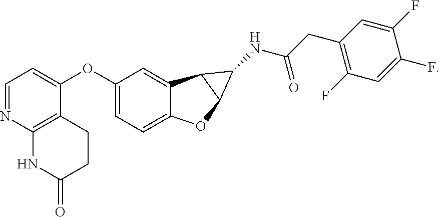 [SMILES: O=C1CCc2c(Oc3ccc4c(c3)[C@H]3[C@H](NC(=O)Cc5cc(F)c(F)cc5F)[C@H]3O4)ccnc2N1]

This compound is also known as 1-((1S,1aS,6bS)-5-((7-oxo-5,6,7,8-tetrahydro-1,8-naphthyridin-4-yl)oxy)-1a,6b-dihydro-1H-cyclopropa[b]benzofuran-1-yl)-3-(2,4,5-trifluorophenyl) urea.

The term “subject” refers to an animal, including, but not limited to, a primate (e.g., human), cow, sheep, goat, horse, dog, cat, rabbit, rat, or mouse. The terms “subject” and “patient” are used interchangeably herein in reference, for example, to a mammalian subject, such as a human subject.

As used herein, the terms “treat,” “treated,” and “treating” mean both therapeutic treatment and prophylactic or preventative measures wherein the object is to prevent or slow down (lessen) an undesired physiological condition, disorder, or disease, or obtain beneficial or desired clinical results. Thus, those in need of treatment include those already diagnosed with or suspected of having the disorder. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment. The term “therapeutically effective amount” is meant to include the amount of a compound that, when administered, is sufficient to prevent development of, or alleviate to some extent, one or more of the symptoms of a disorder, disease, or condition being treated. The term “therapeutically effective amount” also refers to the amount of a compound that is sufficient to elicit the biological or medical response of a cell, tissue, system, animal, or human, which is being sought by a researcher, veterinarian, medical doctor, or clinician.

In certain aspects, a subject is successfully “treated” for cancer, e.g., lung cancer or ovarian cancer, according to the methods of the present invention if the patient shows one or more of the following: a reduction in the number of or complete absence of cancer cells; relief of one or more symptoms associated with the specific cancer; reduced morbidity and mortality; improvement in quality of life; increased progression-free survival (PFS), disease-free survival (DFS), overall survival (OS), metastasis-free survival (MFS), complete response (CR), minimal residual disease (MRD), partial response (PR), stable disease (SD), a decrease in progressive disease (PD), an increased time to progression (TTP), or any combination thereof. In some aspects, nationally or internationally accepted standards of treatment outcomes in a given cancer can be used to determine whether the combination of effective amounts of Compound A, or pharmaceutically acceptable salt thereof, and Compound B, or pharmaceutically acceptable salt thereof, meets any of these particular endpoints (e.g., CR, PFS, PR).

The terms “pharmaceutically acceptable carrier,” “pharmaceutically acceptable excipient,” “physiologically acceptable carrier,” or “physiologically acceptable excipient” refer to a pharmaceutically acceptable material, composition, or vehicle, such as a liquid or solid filler, diluent, excipient, solvent, or encapsulating material. In one aspect, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See Remington: The Science and Practice of Pharmacy, 21^{st} Edition, Lippincott Williams & Wilkins: Philadelphia, P A, 2005; Handbook of Pharmaceutical Excipients, 5^{th} Edition, Rowe et al., Eds., The Pharmaceutical Press and the American Pharmaceutical Association: 2005; and Handbook of Pharmaceutical Additives, 3^{rd} Edition, Ash and Ash Eds., Gower Publishing Company: 2007; Pharmaceutical Preformulation and Formulation, Gibson Ed., CRC Press LLC: Boca Raton, F L, 2004 (incorporated herein by reference). Excipients can include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

The term “pharmaceutical composition,” as used herein, represents a composition containing a compound described herein formulated with a pharmaceutically acceptable excipient, and can be manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); for intrathecal injection; for intracerebroventricular injections; for intraparenchymal injection; or in any other pharmaceutically acceptable formulation.

The term “pharmaceutically-acceptable salts” refers to the relatively non-toxic, inorganic and organic acid addition salts of Compound A or Compound B. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In certain aspects, the compounds of the present invention may contain one or more acidic functional groups and, thus, are capable of forming pharmaceutically acceptable salts with pharmaceutically acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, e.g., Berge et al., supra).

The terms “about” or “approximately” means within a range of an acceptable error for a particular value as determined by one of ordinary skill in the art, which depends in part on how the value is measured or determined. In certain aspects, the term “about” or “approximately” means within 1, 2, 3, or 4 standard deviations. In some aspects, the term “about” or “approximately” means a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

As used herein, the term “administration” refers to the administration of a composition (e.g., a compound or a preparation that includes a compound as described herein) to a subject or system. Administration to an animal subject (e.g., to a human) can be by any appropriate route, such as one described herein.

The terms “drug,” “therapeutic agent,” and “chemotherapeutic agent” refer to a compound, or a pharmaceutical composition thereof, which is administered to a subject for treating, preventing, or ameliorating one or more symptoms of a condition, disorder, or disease.

The terms “co-administration”, “co-administering”, or “co-administered” refer to administering a combination of therapeutic agents, such as, for example, a combination of Compound A, or pharmaceutically acceptable salt thereof, and Compound B, or a pharmaceutically acceptable salt thereof. The combination can be administered as two separate entities, such as, for example, in separate capsules or tablets, or as a single combination entity, such as, for example, in the same capsule or tablet. One therapeutic agent (e.g., Compound A, or a pharmaceutically acceptable salt thereof) can be administered before, concomitantly, or subsequently to the administering of the other therapeutic agent (e.g., Compound B, or pharmaceutically acceptable salt thereof) to the subject.

It is understood that wherever aspects are described herein with the language “comprising,” otherwise analogous aspects described in terms of “consisting of” and/or “consisting essentially of” are also provided.

The details of one or more aspects are set forth in the description below. Other features, objects, and advantages will be apparent from the description and from the claims.

### DETAILED DESCRIPTION OF THE INVENTION

Methods of treating a patient having a solid tumor comprising co-administering to the patient a therapeutically effective amount of Compound A, or a pharmaceutically acceptable salt thereof, and a therapeutically effective amount of Compound B, or a pharmaceutically acceptable salt thereof are disclosed herein.

In some aspects, the present disclosure provides a method of treating a patient having a solid tumor comprising co-administering to the patient a therapeutically effective amount of Compound A or a pharmaceutically acceptable salt thereof, and a therapeutically effective amount of Compound B or a pharmaceutically acceptable salt thereof.

In some aspects, the therapeutically effective amount of Compound A, or pharmaceutically acceptable salt thereof, is about 1 mg to about 10 mg per day. In some aspects, the therapeutically effective amount of Compound A, or pharmaceutically acceptable salt thereof, is about 2 mg to about 8 mg, about 1 mg, about 2 mg, about 3 mg, about 4 mg, about 5 mg, about 6 mg, about 7 mg, about 8 mg, about 9 mg, or about 10 mg per day. In some aspects, the therapeutically effective amount of Compound A, or pharmaceutically acceptable salt thereof, is less than 10 mg, 9 mg, 8 mg, 7 mg, or 6 mg per day.

In some aspects, the therapeutically effective amount of Compound A, or pharmaceutically acceptable salt thereof, is administered in two equal doses. In this aspect, a single dose of Compound A, or a pharmaceutically acceptable salt thereof, is about 0.5 mg to about 5 mg. In some aspect, a single dose of Compound A, or a pharmaceutically acceptable salt thereof, is about 1 mg to about 4 mg, about 0.5 mg, about 1 mg, about 1.5 mg, about 2 mg, about 2.5 mg, about 3 mg, about 3.5 mg, about 4 mg, or about 5 mg.

In some aspects, Compound A is administered in free base form. In some aspects, the therapeutically effective amount of Compound A (as free base) is about 1 mg to about 10 mg per day. In some aspects, the therapeutically effective amount of Compound A (as free base) is about 2 mg to about 8 mg, about 1 mg, about 2 mg, about 3 mg, about 4 mg, about 5 mg, about 6 mg, about 7 mg, about 8 mg, about 9 mg, or about 10 mg per day. In some aspects, the therapeutically effective amount of Compound A (as free base) is less than 10 mg, 9 mg, 8 mg, 7 mg, or 6 mg per day.

In some aspects, the therapeutically effective amount of Compound A (as free base) is administered in two equal doses. In this aspect, a single dose of Compound A (as free base) about 0.5 mg to about 5 mg. In some aspect, a single dose of Compound A (as free base) is about 1 mg to about 4 mg, about 0.5 mg, about 1 mg, about 1.5 mg, about 2 mg, about 2.5 mg, about 3 mg, about 3.5 mg, about 4 mg, or about 5 mg.

In some aspects, the therapeutically effective amount of Compound B, or a pharmaceutically acceptable salt thereof, is about 2 mg to about 50 mg per day. In some aspects, the therapeutically effective amount of Compound B, or a pharmaceutically acceptable salt thereof, is about 2 mg to about 45 mg, 2 mg to about 40 mg, about 5 mg to about 45 mg, or about 5 mg to about 40 mg. In some aspects, the therapeutically effective amount of Compound B, or a pharmaceutically acceptable salt thereof, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, or about 50 mg per day.

In some aspects, Compound B, or a pharmaceutically acceptable salt thereof, is administered once per day.

In some aspects, Compound B is administered in free base form. In some aspects, Compound B is in a pharmaceutically acceptable salt form. In some aspects, the pharmaceutically acceptable salt form is in the form of a hydrochloride salt. In some aspects, the pharmaceutically acceptable salt form is hydroxypropyl methyl cellulose acetate succinate (“HPMCAS”).

In some aspects, the therapeutically effective amount of Compound B is about 2 mg to about 50 mg per day. In some aspects, the therapeutically effective amount of Compound B is about 2 mg to about 45 mg, 2 mg to about 40 mg, about 5 mg to about 45 mg, or about 5 mg to about 40 mg. In some aspects, the therapeutically effective amount of Compound B is about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, or about 50 mg per day.

In some aspects, Compound B is administered once per day.

In some aspects, the therapeutically effective amount of Compound A, or pharmaceutically acceptable salt thereof, is administered in two separate doses of about 2 mg to about 4 mg (for a total daily amount of 4 mg to about 8 mg) and the therapeutically effective amount of Compound B, or a pharmaceutically acceptable salt thereof, is about 5 mg to about 40 mg administered once per day.

In some aspects, the therapeutically effective amount of Compound A (as free base) is administered in two separate doses of about 2 mg to about 4 mg (for a total daily amount of 4 mg to about 8 mg) and the therapeutically effective amount of Compound B is about 5 mg to about 40 mg administered once per day.

In some aspects, Compound A or a pharmaceutically acceptable salt thereof, and/or Compound B, or pharmaceutically acceptable salt thereof, are administered orally.

In some aspects, Compound A or a pharmaceutically acceptable salt thereof, and Compound B or a pharmaceutically acceptable salt thereof, are administered in the same or different dosage forms. In some aspects, Compound A or a pharmaceutically acceptable salt thereof, and Compound B or a pharmaceutically acceptable salt thereof, are administered in different dosage forms. In some aspects, Compound A or a pharmaceutically acceptable salt thereof, is administered before, concomitantly, or subsequently to the administering of Compound B or a pharmaceutically acceptable salt thereof. In some aspects, Compound A or

In some aspects, Compound A or a pharmaceutically acceptable salt thereof, and Compound B or a pharmaceutically acceptable salt thereof, are administered in the same dosage form. In some aspects, the dosage form comprising Compound A or a pharmaceutically acceptable salt thereof, and Compound B or a pharmaceutically acceptable salt thereof, is a capsule.

In some aspects, Compound A or a pharmaceutically acceptable salt thereof, and Compound B or a pharmaceutically acceptable salt thereof, are administered to treat a patient having a solid tumor. In some aspects, the solid tumor is selected from the group consisting of malignant peripheral nerve sheath tumor, biliary tract cancer, breast cancer, cholangiocarcinoma, urothelial cancer, uterine neoplasm, gastric cancer, sarcoma, bladder cancer, head and neck cancer, endometrial cancer, esophageal cancer, adenoid cystic carcinoma, gallbladder cancer, colorectal cancer, thyroid cancer, hepatocellular cancer, prostate cancer, oral cancer, cervical cancer, pancreatic carcinoma, ovarian cancer, melanoma, lung cancer, and serous carcinoma to the peritoneum. In some aspects, the lung cancer is selected from the group consisting of lung adenocarcinoma, squamous non-small cell lung cancer, non-squamous non-small cell lung cancer, and small cell lung cancer. In certain aspects, the patient has non-small cell lung cancer. In some aspects, the patient has non-small cell lung cancer (NSCLC) that is NSCLC with BRAF Class II/III/fusion mutations or KRAS mutant NSCLC.

In certain aspects, the patient has melanoma. In some aspects, the patient has melanoma that is NRAS mutant cutaneous melanoma.

In some aspects, the patient has endometrial cancer. In other aspects, the patient has ovarian cancer. In yet other aspects, the patient has low grade serous ovarian cancer.

In some aspects, the patient has a confirmed mutation in one or more of KRAS, NRAS, HRAS, BRAF, NF1, MEK1, and MEK2.

In certain aspects Compound A, or a pharmaceutically acceptable salt thereof, and Compound B, or a pharmaceutically acceptable salt form thereof, are administered on a 28-day dosing cycle comprising administering Compound A, or a pharmaceutically acceptable salt thereof, twice a day and Compound B, or a pharmaceutically acceptable salt thereof, will be administered once per day. In some aspects, the 28-day dosing cycle is repeated up to a total of 24 consecutive 28-day dosing cycles.

All references (including patents and patent applications) cited herein are hereby incorporated by reference.

### EXAMPLE

It is understood that the example and aspects described herein are for illustrative purposes only and that various modifications or changes in light thereof will be suggested to persons skilled in the art and are to be included within the spirit and purview of this application.

A Phase 1/2a open-label, multicenter, dose escalation and expansion study of Compound A in combination with Compound B adult participants with histologically confirmed, advanced (American Joint Committee on Cancer (AJCC) Stage III or IV) metastatic or unresectable solid cancer that is refractory to or has progressed during or after at least 1 line of appropriate prior systemic anti-cancer therapy including chemotherapy, immunotherapy, or appropriate targeted therapy, or for which there is no treatment available, or prior standard of care therapy was not tolerated will be conducted.

The study will be conducted in two sequential parts: Part 1 dose escalation (Phase 1) and Part 2 dose expansion (Phase 2a).

Participants will receive Compound A (free base) and Compound B administered by mouth every day on a continuous schedule. Compound A (free base) will be dosed twice a day (BID) and Compound B will be dosed once a day (QD). One treatment cycle will be 28 days.

Part 1 (Phase 1 dose escalation): Part 1 of the study will assess the safety, tolerability, pharmacokinetics (PK), pharmacodynamics (PDx), and preliminary evidence of anti-tumor efficacy. Part 1 will also identify the maximum tolerated dose (MTD) and the Recommended Phase 2 dose (RP2D) for the combination of Compound A (free base) with Compound B.

Part 1 Eligible Population: Part 1 will include participants with advanced metastatic or unresectable solid tumors harboring an oncogenic mutation or other aberrations of the MAPK pathway. The mutations and aberrations of the MAP pathway include: a known mutation status and tumor harboring an oncogenic mutation of the BRAF gene (including BRAF Class I or V600 mutation, the BRAF Class II or non-V600 mutation that is kinase-activated, Class III mutation or non-V600 mutation that is kinase-impaired, or BRAF fusion), ARAF, and RAF1/CRAF. In addition, participants with tumors harboring the mutation of NRAS, KRAS, or HRAS, NF1, MAP2K1, MAP2K2, and MAPK1 are eligible for Part 1. Participants with colorectal cancer (CRC) or pancreatic KRAS mutated tumors together will be limited to no more than one third of the participants enrolled in each cohort in Part 1 to increase participant population diversity.

The tumor mutational status will be determined by the Clinical Laboratory Improvement Amendments (CLIA)-certified next generation sequencing (NGS) assay of the archival tumor sample or the fresh tumor biopsy. This information may be available from historic reports obtained at any time before the start of the study treatments or from the assay at the local laboratory performed during screening.

Part 1 Dose Escalation Plan: The planned dose levels of Compound A (free base) and Compound B in Part 1 are shown below:
Cohort 1: Compound A (free base) at 2 mg BID+Compound B at 5 mg QD
Cohort 2: Compound A (free base) at 2 mg BID+Compound B at 10 mg QD
Cohort 3: Compound A (free base) at 2 mg BID+Compound B at 20 mg QD
Cohort 4: Compound A (free base) at 2 mg BID+Compound B at 30 mg QD
Cohort 5: Compound A (free base) at 3 mg BID+Compound B at 30 mg QD
Cohort 6: Compound A (free base) at 4 mg BID+Compound B at 30 mg QD

If the highest planned dose level (Cohort 6) is cleared by the Cohort Management Committee (CMC) based on the Bayesian Optimal Interval (BOIN) estimate of the toxicity rate and the review of all available data in the study, then the Sponsor in consultation with the CMC may continue dose escalation. The highest doses of Compound A (as free base) and Compound B in Part 1 dose escalation will not exceed 8 mg BID and 40 mg QD, respectively.

If a planned dose level is not tolerated based on the Bayesian Optimal Interval (BOIN) estimate of the toxicity rate derived from the observed dose-limiting toxicity (DLT) rate and/or the review of all available data in the study, an alternate dose exploration may be implemented to identify the optimal dose ratio for the combination of agents. An alternate dose finding scheme may explore lowering of the Compound B dose while escalating the Compound A (as free base) dose.

After the dose escalation is completed, the MTD will be computed using an isotonic regression. Specifically, the MTD is selected as the dose for which the isotonic estimate of the toxicity rate is less than or equal to 0.359 and closest to the target toxicity rate.

The non-tolerable dose is defined as the dose of study treatments where the target DLT rate was exceeded.

The dose escalation will be stopped either when the non-tolerable dose is reached or the highest planned dose level of Compound A (as free base) or Compound B (8 mg BID or 40 mg QD, respectively) is shown to be tolerated.

Part 1 RP2D selection: Based on the evaluation of all available study data from Part 1, a dose and dosing schedule to be used as the RP2D for Compound A (as free base) and Compound B in Part 2 will be recommended.

Part 2 (Phase 2a dose expansion): Part 2 will begin after the RP2D for the combination of Compound A (as free base) and Compound B is identified in Part 1. Part 2 may start either in parallel with, or after, the conduct and analysis of the PDx Expansion Cohort in Part 1.

Part 2 will confirm the safety, tolerability, efficacy, PK, and PDx for the combination of Compound A (as free base) and Compound B.

Part 2 will follow a parallel design and include one or more dose expansion cohorts, where each participant would be treated with the combination of Compound A (as free base) and Compound B at the RP2D.

Participants who experience a treatment emergent Adverse Events (TEAE) requiring treatment modification will be managed according to the applicable guidelines in this protocol.

Part 2 Eligible Populations: The populations eligible for Part 2 will include participants with advanced metastatic or unresectable solid tumors harboring the oncogenic mutations specific for each of the expansion cohorts. The tumor mutational status should be determined by CLIA-compliant NGS assay of the archival tumor sample or the fresh tumor biopsy. This information may be available from historic reports obtained at any time before the start of the study treatments or from the assay at the local laboratory performed during screening.

One or more of the following cohorts may be opened in parallel, or sequentially at the Sponsor's discretion:
1. Expansion Cohort A (n=20 evaluable participants): Participants with cutaneous melanoma harboring an NRAS mutation.
   a. Minimum of 15 (75%) of the 20 participants in Cohort A must have a NRAS Q61× mutation (i.e., Q61R, Q61K, Q61L).
2. Expansion Cohort B (n=30 evaluable participants): Participants with NSCLC harboring a KRAS mutation
   a. Minimum of 15 (50%) of the 30 evaluable participants in Cohort B must have a KRAS G12V mutation.
3. Expansion Cohort C (n=30 evaluable participants): Participants with NSCLC or cutaneous melanoma harboring BRAF Class II or Class III mutations or BRAF fusion.
   a. Cohort C1: Approximately 15 evaluable participants with NSCLC harboring BRAF Class II or Class III mutations or BRAF fusion.
   b. Cohort C2: Approximately 15 evaluable participants with cutaneous melanoma harboring BRAF Class II or Class III mutations or BRAF Fusion.

1. A method of treating a patient having a solid tumor comprising co-administering to the patient a therapeutically effective amount of mirdametinib, or a pharmaceutically acceptable salt thereof, and a therapeutically effective amount of 1-((1S,1aS,6bS)-5-((7-oxo-5,6,7,8-tetrahydro-1,8-naphthyridin-4-yl)oxy)-1a,6b-dihydro-1H-cyclopropa[b]benzofuran-1-yl)-3-(2,4,5-trifluorophenyl) urea, or a pharmaceutically acceptable salt thereof.

2. The method of claim 1, wherein the therapeutically effective amount of mirdametinib or a pharmaceutically acceptable salt thereof is about 1 mg to about 10 mg per day.

3. The method of claim 1, wherein the therapeutically effective amount of 1-((1S,1aS,6bS)-5-((7-oxo-5,6,7,8-tetrahydro-1,8-naphthyridin-4-yl)oxy)-1a,6b-dihydro-1H-cyclopropa[b]benzofuran-1-yl)-3-(2,4,5-trifluorophenyl) urea, or a pharmaceutically acceptable salt thereof, is about 2 mg to about 50 mg per day.

4. The method of claim 1, wherein the therapeutically effective amount of mirdametinib is a total amount of up to 10 mg per day and the therapeutically effective amount of 1-((1S,1aS,6bS)-5-((7-oxo-5,6,7,8-tetrahydro-1,8-naphthyridin-4-yl)oxy)-1a,6b-dihydro-1H-cyclopropa[b]benzofuran-1-yl)-3-(2,4,5-trifluorophenyl) urea, or a pharmaceutically acceptable salt thereof, is about 5 mg to about 40 mg per day.

5. The method of claim 4, wherein the therapeutically effective amount of mirdametinib is administered in two equal doses.

6. The method of claim 1, wherein 1-((1S,1aS,6bS)-5-((7-oxo-5,6,7,8-tetrahydro-1,8-naphthyridin-4-yl)oxy)-1a,6b-dihydro-1H-cyclopropa[b]benzofuran-1-yl)-3-(2,4,5-trifluorophenyl) urea is in a pharmaceutically acceptable salt form.

7. The method of claim 6, wherein the pharmaceutically acceptable salt form is a hydroxypropyl methyl cellulose acetate succinate salt.

8. The method of claim 1, wherein mirdametinib, or pharmaceutically salt thereof, and 1-((1S,1aS,6bS)-5-((7-oxo-5,6,7,8-tetrahydro-1,8-naphthyridin-4-yl)oxy)-1a,6b-dihydro-1H-cyclopropa[b]benzofuran-1-yl)-3-(2,4,5-trifluorophenyl) urea, or a pharmaceutically acceptable salt thereof, are administered in the same or different dosage forms.

9. The method of claim 8, wherein mirdametinib is administered in free base form.

10. The method of claim 1, wherein 1-((1S,1aS,6bS)-5-((7-oxo-5,6,7,8-tetrahydro-1,8-naphthyridin-4-yl)oxy)-1a,6b-dihydro-1H-cyclopropa[b]benzofuran-1-yl)-3-(2,4,5-trifluorophenyl) urea, or a pharmaceutically acceptable salt thereof, is administered once per day.

11. The method of claim 10, wherein mirdametinib, or a pharmaceutically acceptable salt thereof, is administered before, concomitantly, or subsequently to the administering of 1-((1S,1aS,6bS)-5-((7-oxo-5,6,7,8-tetrahydro-1,8-naphthyridin-4-yl)oxy)-1a,6b-dihydro-1H-cyclopropa[b]benzofuran-1-yl)-3-(2,4,5-trifluorophenyl) urea, or a pharmaceutically acceptable salt thereof.

12. The method of claim 1, wherein mirdametinib, or a pharmaceutically acceptable salt thereof, and 1-((1S,1aS,6bS)-5-((7-oxo-5,6,7,8-tetrahydro-1,8-naphthyridin-4-yl)oxy)-1a,6b-dihydro-1H-cyclopropa[b]benzofuran-1-yl)-3-(2,4,5-trifluorophenyl) urea, or a pharmaceutically acceptable salt thereof, are administered orally.

13. The method of claim 1, wherein the solid tumor is selected from the group consisting of malignant peripheral nerve sheath tumor, biliary tract cancer, breast cancer, cholangiocarcinoma, urothelial cancer, uterine neoplasm, gastric cancer, sarcoma, bladder cancer, head and neck cancer, endometrial cancer, esophageal cancer, adenoid cystic carcinoma, gallbladder cancer, colorectal cancer, thyroid cancer, hepatocellular cancer, prostate cancer, oral cancer, cervical cancer, pancreatic carcinoma, ovarian cancer, melanoma, lung cancer, and serous carcinoma to the peritoneum.

14. The method of claim 13, wherein the lung cancer is selected from the group consisting of lung adenocarcinoma, squamous non-small cell lung cancer, non-squamous non-small cell lung cancer, and small cell lung cancer.

15. The method of claim 14, wherein the patient has non-small cell lung cancer.

16. The method of claim 15, wherein the patient has non-small cell lung cancer (NSCLC) that is NSCLC with BRAF Class II/III/fusion mutations or KRAS mutant NSCLC.

17. The method of claim 13, wherein the patient has melanoma.

18. The method of claim 17, wherein the patient has melanoma that is NRAS mutant cutaneous melanoma.

19. The method of claim 13, wherein the patient has endometrial cancer.

20. The method of claim 13, wherein the patient has ovarian cancer.

21. The method of claim 13, where the patient has low grade serous ovarian cancer.

22. The method of claim 1, wherein the patient has a confirmed mutation in one or more of KRAS, NRAS, HRAS, BRAF, NF1, MEK1, and MEK2.

23. The method of claim 1, wherein mirdametinib, or a pharmaceutically acceptable salt thereof, and 1-((1S,1aS,6bS)-5-((7-oxo-5,6,7,8-tetrahydro-1,8-naphthyridin-4-yl)oxy)-1a,6b-dihydro-1H-cyclopropa[b]benzofuran-1-yl)-3-(2,4,5-trifluorophenyl) urea, or a pharmaceutically acceptable salt thereof, are administered on a 28-day dosing cycle comprising administering mirdametinib, or a pharmaceutically acceptable salt thereof, twice a day and 1-((1S,1aS,6bS)-5-((7-oxo-5,6,7,8-tetrahydro-1,8-naphthyridin-4-yl)oxy)-1a,6b-dihydro-1H-cyclopropa[b]benzofuran-1-yl)-3-(2,4,5-trifluorophenyl) urea, or a pharmaceutically acceptable salt thereof, once per day.

24. The method of claim 23, wherein the 28-day dosing cycle is repeated up to a total of 24 consecutive 28-day dosing cycles.

25. The method of claim 1, wherein mirdametinib, or pharmaceutically acceptable salt thereof, and/or 1-((1S,1aS,6bS)-5-((7-oxo-5,6,7,8-tetrahydro-1,8-naphthyridin-4-yl)oxy)-1a,6b-dihydro-1H-cyclopropa[b]benzofuran-1-yl)-3-(2,4,5-trifluorophenyl) urea, or pharmaceutically acceptable salt thereof, are administered in tablet and/or capsule form.
